(19) United States
(12) Patent Application Publication
(10) Pub. No.: US 2012/0067715 A1
(43) Pub. Date: Mar. 22, 2012

(54) METHOD FOR PURIFYING CARBOXYLIC ACIDS CONTAINING HALOGEN COMPOUNDS

(75) Inventors: Guenther Forster, Ludwigshafen (DE); Vijay Narayanan Swaminathan, Ludwigshafen (DE); Franz Niklaus Windlin, Heidelberg (DE); Thomas Leiendecker, Gau-Weinheim (DE); Sebastian Peter Smidt, Offesheim (DE)

(73) Assignee: BASF SE, Ludwigshafen (DE)

(21) Appl. No.: 13/321,405

(22) PCT Filed: May 19, 2010
(86) PCT No.: PCT/EP2010/056927

§ 371 Date: Nov. 18, 2011

(30) Foreign Application Priority Data
May 20, 2009 (EP) 09160841.4

(51) Int. Cl. C07C 51/44; B01D 3/34

(52) U.S. Cl. 203/58; 203/50

(57) ABSTRACT

In a process for purifying carboxylic acids comprising halogen compounds, the carboxylic acid is distilled in the presence of a nonvolatile auxiliary base, the halide of which is liquid at the boiling temperature of the carboxylic acid. The auxiliary base binds the hydrogen halide which is present in the carboxylic acid and/or has been eliminated from halogen compounds as a result of thermal action and lowers the vapor pressure thereof in this way, such that the hydrogen halide is held in the distillation bottoms and is not transferred into the distillate. Since the halide of the auxiliary base is liquid, the formation of solid deposits in the distillation apparatus is prevented. The carboxylic acid optionally also comprises at least one low boiler.

The present invention relates to a process for purifying carboxylic acids comprising halogen compounds.

Acylations with carboxylic anhydrides, such as acetic anhydride, are processes performed frequently in the chemical industry. In this acylation, one molecule of carboxylic acid is obtained as a by-product per acyl group introduced into the target molecule. It is desirable to recover the carboxylic acid obtained and to send it to a further use. This use of the carboxylic acid is, however, made more difficult in many cases by impurities in the form of halogen compounds which originate from the halogenated Lewis catalysts, with which the acylation and/or preceding conversions are catalyzed, and optionally low boilers which originate from the solvents used.

For example, EP-A 0087576 describes the esterification of tocopherol with acetic anhydride or propionic anhydride to tocopheryl acetate or tocopheryl propionate in the presence of zinc chloride and of a liquid hydrocarbon as solvents. Suitable solvents are hydrocarbons such as hexane, heptane, cyclohexane, benzene or toluene.

There is therefore a need for a process for purifying carboxylic acids comprising halogen compounds.

DE-A 211 97 44 discloses a process for purifying a carboxylic acid contaminated by halogenated materials, in which a contaminated carboxylic acid stream is introduced into a first distillation column between the ends thereof, a product stream is withdrawn from the upper part of the first column and this is introduced into a second column between the ends thereof and a carboxylic acid stream is withdrawn from the lower part of the second column, this carboxylic acid stream being essentially free of halogenated material, and an overhead fraction of the second column is removed, which comprises halogenated materials.

GB 850 960 describes a process for obtaining monocarboxylic acids from mixtures of monocarboxylic acids and brominated compounds, in which the mixtures are distilled in the presence of a metal compound which prevents volatilization of the brominated compounds.

EP-A 0 135 085 discloses a process for removing iodine and compounds thereof from the carbonylation products obtained in the carbonylation of dimethyl ether, methyl acetate or methanol, said products being acetic acid, acetic anhydride or ethylidene diacetate. The carbonylation products are treated at temperatures of 20 to 250° C. with alkyl- or arylphosphines or heterocyclic aromatic nitrogen compounds and at least one of the metals copper, silver, zinc or cadmium or compounds thereof, and the carbonylation products are removed by distillation from the iodine bound in nonvolatile form as a result.

EP-A 0 545 101 discloses a process for purifying waste acetic acid, wherein the waste acetic acid is admixed in a first step with a complex-forming metal or one of the compounds thereof and a basic compound, and this mixture is kept at a temperature between 25 and 118° C. over a period of 1 to 6 hours, a first fraction is distilled off in a second step, and purified acetic acid is distilled off as the top product from a nonvolatile still residue in a third step.

The known processes in which inorganic bases and/or transition metal compounds are used in order to bind halogen compounds in the distillation residue have the disadvantage that solid deposits can form in the distillation apparatus, which impair the trouble-free operation of the distillation apparatus.

The invention provides a process for purifying carboxylic acids comprising halogen compounds, in which the carboxylic acid is distilled in the presence of a nonvolatile auxiliary base, the halide of which is liquid at the boiling temperature of the carboxylic acid.

The distillation is preferably effected in the absence of transition metal compounds and preferably in the absence of inorganic bases.

The halide of the auxiliary base is understood to mean the reaction product of the auxiliary base with a hydrogen halide. Since the halide of the auxiliary base is liquid under the process conditions, the formation of solid deposits in the distillation apparatus is prevented and the withdrawal of the halide in liquid form from the bottom vessel of the distillation apparatus is enabled. These are crucial advantages in the distillation.

In the present context, halogen is understood to mean fluorine (F), chlorine (Cl), bromine (Br) or iodine (I), preferably chlorine or bromine, especially chlorine.

The halogen compounds for removal in accordance with the invention comprise (free) hydrogen halide and organic halogen compounds, especially α-halocarboxylic acids. It will be appreciated that hydrogen halides can dissociate to protons and halide ions in an equilibrium reaction. For the present purposes, halide ions are therefore also counted as hydrogen halides. The process according to the invention is applicable particularly to carboxylic acids which comprise hydrogen halide, optionally in combination with organic halogen compounds.

While the depletion of the hydrogen halide content by formation of the halide of the auxiliary base used can be explained in a simple manner, the observed depletion of the organic halogen compounds has not been clarified in detail. It is probable that the thermal stress decomposes the organic halogen compounds in the bottom of the distillation apparatus to hydrogen halide and halogen-free components. The hydrogen halide formed can in turn be scavenged by the auxiliary base. Organic halogen compounds in the form of α-halocarboxylic acids also have a higher acidity than (halogen-free) carboxylic acids and therefore preferentially form salts with the auxiliary base. For example, chloroacetic acid and dichloroacetic acid are more acidic than acetic acid and are preferentially deprotonated, and so are retained as nonvolatile acid in the bottoms.

The process according to the invention is suitable for purifying all carboxylic acids which are distillable essentially without decomposition.

The carboxylic acid to be purified is preferably liquid at 25° C.

These include aliphatic monocarboxylic acids, especially aliphatic monocarboxylic acids having 1 to 10 carbon atoms such as formic acid, acetic acid, propionic acid, butyric acid, valeric acid, pivalic acid, caproic acid, ethylhexanoic acid, propylheptanoic acid, isononanoic acid and cyclopentanecarboxylic acid.

Among these, formic acid and acetic acid are the most preferred.

The content of halogen compounds (calculated as halogen) in the carboxylic acid to be purified may generally be up to 5% by weight, usually up to 1% by weight, generally up to 0.1% by weight. The content of halogen compounds (calculated as halogen) in the carboxylic acid purified by the process according to the invention is generally less than 100 ppm, preferably less than 20 ppm, especially less than 5 ppm. The total content of halogen compounds can be determined by methods of elemental analysis known to those skilled in the art, for example by coulometric determination by means of combustion (for example with a Euroglas ECS 1200 TOX Analyzer (Thermo Electron GmbH, Dreieich, Germany); cf., for example, F. Ehrenberger “Quantitative organische Elementaranalyse” [Quantitative organic elemental analysis] ISBN 3-527-28056-1 or DIN 51408 part 2 “Bestimmung des Chlorgehaltes” [Determination of the chlorine content]). Hydrogen halide (including halide ions) can be determined by quantitative analysis by means of silver nitrate (argentometry). The proportion of organic halogen compounds can be determined as the difference between total halogen and hydrogen halide (including halide ions). When the constitution of the contaminating organic halogen compounds is known, a determination by mass spectrometry is also an option.

In one embodiment of the process according to the invention, the carboxylic acid also comprises at least one low boiler. Low boilers are understood to mean compounds with a boiling temperature below the boiling temperature of the carboxylic acid to be purified. The contamination of the carboxylic acid by low boilers generally originates from organic solvents in which chemical reactions, for example acylations with carboxylic anhydrides, are performed, the by-product of which is the carboxylic acid which is the starting material of the process according to the invention.

Examples of possible low boilers include:

aliphatic hydrocarbons such as n-pentane, pentane isomers and mixtures thereof, n-hexane, hexane isomers and mixtures thereof, n-heptane, heptane isomers and mixtures thereof, n-octane, octane isomers and mixtures thereof, isooctane, cyclohexane, methylcyclohexane, decalin;
aromatic hydrocarbons such as benzene, toluene, o-xylene, m-xylene, p-xylene and mixtures thereof;
halogenated hydrocarbons such as dichloromethane, trichloromethane, 1,2-dichloroethane, 1,2-dichloroethene, 1,1,1-trichloroethane;
ethers such as dimethyl ether, diethyl ether, tert-butyl methyl ether (MTBE), dioxane, tetrahydrofuran;
esters such as methyl acetate, ethyl acetate;
ketones such as acetone, ethyl methyl ketone, cyclohexanone.

The maximum content of low boilers in the carboxylic acid to be purified corresponds to the amount of low boilers which is soluble homogeneously in the carboxylic acid, generally up to 50% by weight, usually up to 3% by weight or up to 1% by weight. The content of low boilers in the carboxylic acid purified by the process according to the invention is generally less than 1% by weight, preferably less than 0.5% by weight, especially less than 0.1% by weight.

The distillation can be performed at reduced pressure, standard pressure or elevated pressure. A preferred pressure range is 15 mbar to 1 bar, preferably 200 to 600 mbar. The distillation can be performed within a temperature range (bottom temperature) from 20 to 250° C., preferably at least 70° C.

If impurities other than halogen compounds are absent or the removal of impurities other than halogen compounds is not desired, the distillation, given a sufficient boiling point difference between carboxylic acid and auxiliary base, can be performed as a simple distillation, i.e. essentially without mass transfer between vapor and condensate.

Preference is given to performing the distillation, however, as a fractional distillation in one or more, such as 2 or 3, distillation apparatuses. Useful apparatus for the distillation is that customary therefor, as described, for example, in Kirk-Othmer, Encyclopedia of Chemical Technology, 3rd ed., vol. 7, John Wiley & Sons, New York, 1979, pages 870-881.

The distillation is preferably effected in one or more columns with internals which consist of trays, rotating internals, and random and/or structured packings.

Useful column trays include (i) trays with bores or slots in the tray plate; (ii) trays with necks or chimneys covered by bubble-caps, caps or hoods; (iii) trays with bores in the tray plate, which are covered by movable valves; (iv) trays with special constructions.

In columns with rotating internals, the return stream is either sprayed by rotating funnels or spread out with the aid of a rotor as a film on a heated tube wall.

Columns used in the process according to the invention may have random beds with various random packings. They may consist of all suitable materials, such as steel, stainless steel, nickel-base alloys such as HC, copper, carbon, stoneware, porcelain, glass, plastics, and be present in various forms, such as spheres, rings with smooth or profiled surfaces, rings with internal elements or wall breaches, wire mesh rings, saddles and spirals.

Packings with regular geometry may consist, for example, of metal sheets or fabrics. Examples of such packings are Sulzer BX fabric packings made of metal or plastic, Sulzer Mellapack lamellar packings composed of sheet metal, structured packings from Sulzer (Optiflow), Montz (BSH) and Kühni (Rombopack).

The distillation column(s) is (are) provided with devices for bottom heating. Useful devices for this purpose include evaporators incorporated into the bottom, for example a Robert evaporator, or a circulation system with an external evaporator, for example tube or plate heat exchangers. A circulation system is then, for example, force circulation or natural circulation. The distillation column(s) is (are) generally also provided with devices for condensing and collecting the top product. A condensate divider can be used to introduce a portion of the top condensate as a return stream back to the columns.

When the carboxylic acid to be purified comprises low boilers, the distillation can be performed in columns connected in series, in which case a low boiler fraction is first drawn off at the top in a low boiler column and a bottom discharge from the low boiler column is passed into a pure carboxylic acid column in which the pure carboxylic acid is removed from a high-boiling bottom residue. The low boiler fraction may consist of essentially pure low boilers and/or a low boiler-carboxylic acid azeotrope.

In an embodiment which is simpler in apparatus terms and therefore preferred, the mixture of the carboxylic acid to be purified and of the auxiliary base is introduced into a distillation column between the top and bottom thereof. The distillation column comprises a column section above the feed, designed as a rectifying section with a plurality of theoretical plates, and a column section below the feed, designed as a stripping section. Low boilers are drawn off at the top of the distillation column, for example as pure low boilers and/or in the form of a low boiler-carboxylic acid azeotrope. The pure carboxylic acid is drawn off in gaseous form from the lower region, the bottom or the circulation system of the bottom heater of the distillation column. The auxiliary base and the halide of the auxiliary base collect in the bottom.

The mixture of the carboxylic acid to be purified and of the auxiliary base can appropriately be converted in a preliminary reactor before being introduced into the distillation column, in order to complete the reaction between the hydrogen halide and the auxiliary base and/or thermal decomposition reactions of organic halogen compounds.

This embodiment is particularly suitable, for example, when the carboxylic acid is acetic acid and the low boiler is heptane (including heptane isomer mixtures).

According to the invention, the distillation of the carboxylic acid is effected in the presence of a nonvolatile auxiliary base. In the present context, the expression “nonvolatile” shall mean that the boiling temperature of the auxiliary base at the pressure at which the distillation is performed is higher than that of the carboxylic acid, preferably at least 35° C. higher, especially at least 50° C. higher, more preferably at least 75° C., than the boiling temperature of the carboxylic acid.

The auxiliary base is selected such that the halide of the auxiliary base is liquid at the boiling temperature of the carboxylic acid. Such liquid salts are often referred to as ionic liquids.

The auxiliary base binds the hydrogen halide present in the carboxylic acid and/or eliminated from halogen compounds by thermal action, and in this way lowers the vapor pressure thereof, such that the hydrogen halide is held in the distillation bottoms and is not transferred to the distillate.

The auxiliary base is used in a stoichiometric amount or in a stoichiometric excess (calculated as neutralization equivalents of the auxiliary base), based on the halogen compounds present in the carboxylic acid to be purified (calculated as halogen atoms). A neutralization equivalent is understood to mean the imaginary fraction of the base that can absorb a proton. The auxiliary base is used generally in an amount of 1 to 30 equivalents, preferably 1 to 2 equivalents (calculated as neutralization equivalents), based on the halogen compounds present (calculated as halogen atoms).

The compounds usable as auxiliary bases may comprise phosphorus, sulfur or nitrogen atoms, for example at least one nitrogen atom, preferably one to ten nitrogen atoms, more preferably one to five, even more preferably one to three and especially one to two nitrogen atoms. Optionally, further heteroatoms, for example oxygen, sulfur or phosphorus atoms, may be present.

Preference is given to those compounds which comprise at least one five- to six-membered heterocycle which has at least one nitrogen atom and optionally an oxygen or sulfur atom, particular preference to those compounds which comprise at least one five- to six-membered heterocycle which has one, two or three nitrogen atoms and a sulfur atom or an oxygen atom, very particular preference to those having two nitrogen atoms.

Particularly preferred compounds are those which have a molar mass less than 1000 g/mol, even more preferably less than 500 g/mol and especially less than 250 g/mol.

Additionally preferred are those compounds which are usable as bases and are selected from the compounds of the formulae (Ia) to (Ir)

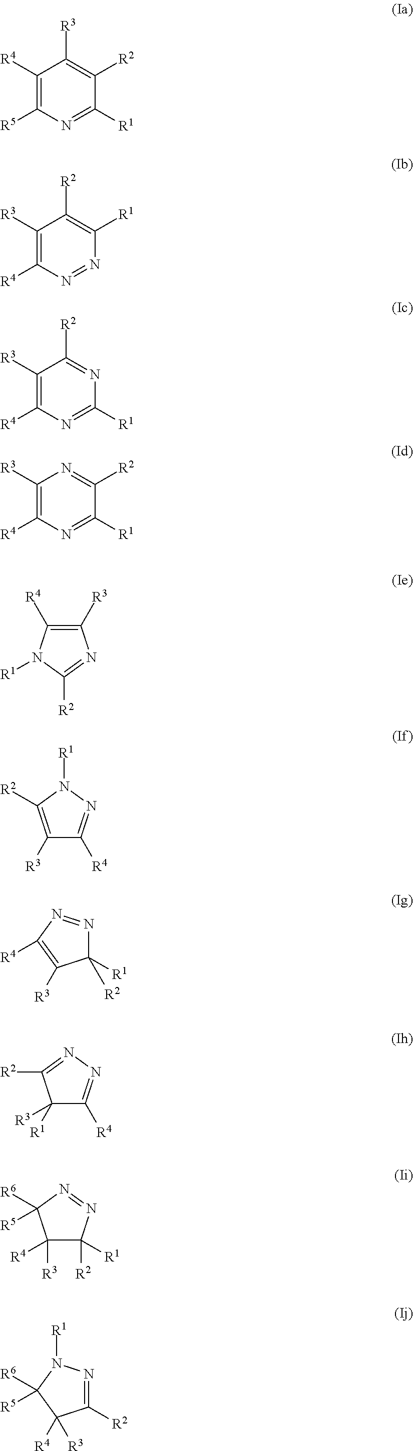

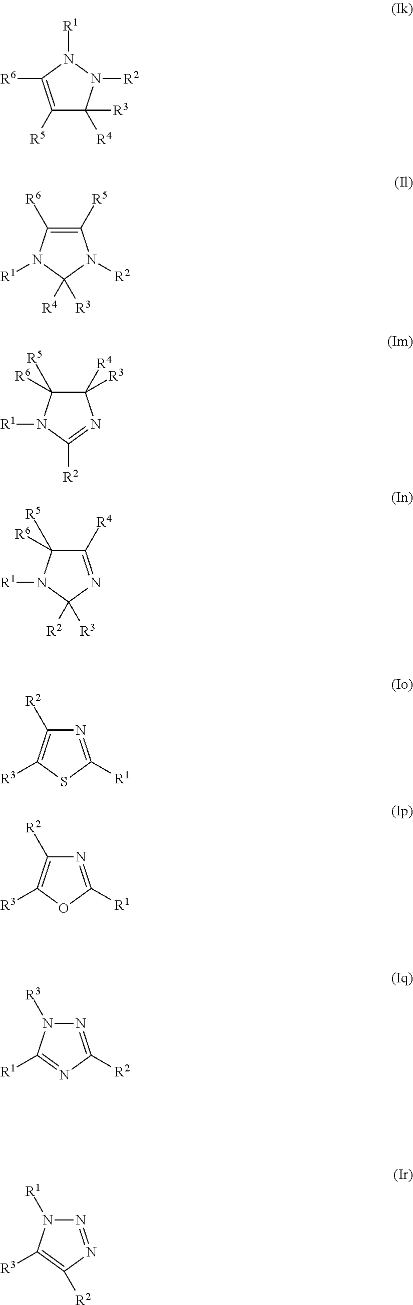

and oligo- or polymers which comprise these structures,
in which
R², R³, R⁴, R⁵ and R⁶ are each independently hydrogen, C₁-C₁₈-alkyl, C₂-C₁₈-alkyl which is optionally interrupted by one or more oxygen and/or sulfur atoms and/or one or more substituted or unsubstituted imino groups, C₆-C₁₂-aryl, C₅-C₁₂-cycloalkyl or a five- to six-membered heterocycle having oxygen, nitrogen and/or sulfur atoms, or two of them together form an unsaturated, saturated or aromatic ring optionally interrupted by one or more oxygen and/or sulfur atoms and/or one or more substituted or unsubstituted imino groups, where the radicals mentioned may each be substituted by functional groups, aryl, alkyl, aryloxy, alkyloxy, halogen, heteroatoms and/or heterocycles.

In these radicals,

C₁-C₁₈-alkyl optionally substituted by functional groups, aryl, alkyl, aryloxy, alkyloxy, halogen, heteroatoms and/or heterocycles is, for example, methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hexadecyl, octadecyl, 1,1-dimethyl-propyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di-(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, butylthiomethyl, 2-dodecylthioethyl, 2-phenylthioethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl, and
C₂-C₁₈-alkyl optionally interrupted by one or more oxygen and/or sulfur atoms and/or one or more substituted or unsubstituted imino groups is, for example, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-oxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

When two radicals form a ring, these radicals together may be 1,3-propylene, 1,4-butylene, 2-oxa-1,3-propylene, 1-oxa-1,3-propylene, 2-oxa-1,3-propylene, 1-oxa-1,3-propenylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The number of oxygen and/or sulfur atoms and/or imino groups is unlimited. In general, it is not more than 5 in the radical, preferably not more than 4 and most preferably not more than 3.

In addition, there is generally at least one carbon atom, preferably at least two, between two heteroatoms.

Substituted and unsubstituted imino groups may, for example, be imino, methylimino, isopropylimino, n-butylimino or tert-butylimino.

In addition,

functional groups are carboxyl, carboxamide, hydroxyl, di-(C₁-C₄-alkyl)amino, C₁-C₄-alkyloxycarbonyl, cyano or C₁-C₄-alkyloxy,
C₆-C₁₂-aryl optionally substituted by functional groups, aryl, alkyl, aryloxy, alkyloxy, halogen, heteroatoms and/or heterocycles is, for example, phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxymethylphenyl,
C₅-C₁₂-cycloalkyl optionally substituted by functional groups, aryl, alkyl, aryloxy, alkyloxy, halogen, heteroatoms and/or heterocycles is, for example, cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl, and a saturated or unsaturated bicyclic system, for example norbornyl or norbornenyl,
a five- to six-membered heterocycle having oxygen, nitrogen and/or sulfur atoms is, for example, furyl, thiophenyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl, difluoropyridyl, methylthiophenyl, isopropylthiophenyl or tert-butylthiophenyl, and
C₁ to C₄-alkyl is, for example, methyl, ethyl, propyl, isopropyl, n-butyl, sec-butyl or tert-butyl.

Preferably, R¹, R², R³, R⁴, R⁵ and R⁶ are each independently hydrogen, methyl, ethyl, n-butyl, 2-hydroxyethyl, 2-cyanoethyl, 2-(methoxycarbonyl)ethyl, 2-(ethoxycarbonyl)ethyl, 2-(n-butoxycarbonyl)ethyl, dimethylamino, diethylamino and chlorine.

Particularly preferred pyridines (Ia) are those in which one of the R¹ to R⁵ radicals is methyl, ethyl or chlorine and all others are hydrogen, or R³ is dimethylamino and all others are hydrogen, or all are hydrogen, or R² is carboxyl or carboxamide and all others are hydrogen, or R¹ and R² or R² and R³ are 1,4-buta-1,3-dienylene and all others are hydrogen.

Particularly preferred pyridazines (Ib) are those in which one of the R¹ to R⁴ radicals is methyl or ethyl and all others are hydrogen, or all are hydrogen.

Particularly preferred pyrimidines (Ic) are those in which R² to R⁴ are each hydrogen or methyl and R¹ is hydrogen, methyl or ethyl, or R² and R⁴ are each methyl, R³ is hydrogen and R¹ is hydrogen, methyl or ethyl.

Particularly preferred pyrazines (Id) are those in which R¹ to R⁴ are all methyl or all hydrogen.

Particularly preferred imidazoles (Ie) are those in which R¹ is each independently selected from methyl, ethyl, n-propyl, n-butyl, n-pentyl, n-octyl, 2-hydroxyethyl and 2-cyanoethyl, and

R² to R⁴ are each independently hydrogen, methyl or ethyl.

Particularly preferred 1H-pyrazoles (If) are those in which, each independently,

R¹ is selected from hydrogen, methyl and ethyl,
R², R³ and R⁴ from hydrogen and methyl.

Particularly preferred 3H-pyrazoles (Ig) are those in which, each independently,

R¹ is selected from hydrogen, methyl and ethyl,
R², R³ and R⁴ from hydrogen and methyl.

Particularly preferred 4H-pyrazoles (Ih) are those in which, each independently,

R¹ to R⁴ are selected from hydrogen and methyl.

Particularly preferred 1-pyrazolines (Ii) are those in which, each independently,

R¹ to R⁶ are selected from hydrogen and methyl.

Particularly preferred 2-pyrazolines (ID are those in which, each independently,

R¹ is selected from hydrogen, methyl, ethyl and phenyl, and
R² to R⁶ from hydrogen and methyl.

Particularly preferred 3-pyrazolines (Ik) are those in which, each independently,

R¹ or R² is selected from hydrogen, methyl, ethyl and phenyl, and
R³ to R⁶ from hydrogen and methyl.

Particularly preferred imidazolines (II) are those in which, each independently,

R¹ or R² is selected from hydrogen, methyl, ethyl, n-butyl and phenyl, and
R³ or R⁴ from hydrogen, methyl and ethyl, and
R⁵ or R⁶ from hydrogen and methyl.

Particularly preferred imidazolines (Im) are those in which, each independently,

R¹ or R² is selected from hydrogen, methyl and ethyl, and
R³ to R⁶ from hydrogen and methyl.

Particularly preferred imidazolines (In) are those in which, each independently,

R² or R³ is selected from hydrogen, methyl and ethyl, and
R⁴ to R⁶ from hydrogen and methyl.

Particularly preferred thiazoles (Io) or oxazoles (Ip) are those in which, each independently,

R¹ is selected from hydrogen, methyl, ethyl and phenyl, and
R² or R³ from hydrogen and methyl.

Particularly preferred 1,2,4-triazoles (Iq) are those in which, each independently,

R¹ or R² is selected from hydrogen, methyl, ethyl and phenyl, and
R³ from hydrogen, methyl and phenyl.

Particularly preferred 1,2,3-triazoles (Ir) are those in which, each independently,

R¹ is selected from hydrogen, methyl and ethyl and
R² or R³ from hydrogen and methyl, or
R² and R³ are 1,4-buta-1,3-dienylene and all others are hydrogen.

Among these, the pyridines and the imidazoles are preferred.

Very particularly preferred bases are 3-chloropyridine, 4-dimethylaminopyridine, 2-ethyl-4-aminopyridine, 2-methylpyridine (α-picoline), 3-methylpyridine (β-picoline), 4-methylpyridine (γ-picoline), 2-ethylpyridine, 2-ethyl-6-methylpyridine, quinoline, isoquinoline, 1-C₁-C₄-alkylimidazole, 1-methylimidazole, 1,2-dimethylimidazole, 1-n-butylimidazole, 1,4,5-trimethylimidazole, 1,4-dimethylimidazole, imidazol, 2-methylimidazole, 1-butyl-2-methylimidazole, 4-methylimidazole, 1-n-pentylimidazole, 1-n-hexylimidazole, 1-n-octylimidazole, 1-(2′-aminoethyl)imidazole, 2-ethyl-4-methylimidazole, 1-vinylimidazole, 2-ethylimidazole, 1-(2′-cyanoethyl)imidazole and benzotriazole.

Especially preferred are 1-n-butylimidazole, 1-methylimidazole, 2-methylpyridine and 2-ethylpyridine.

Additionally suitable are tertiary amines of the formula (XI),

NR^{a}R^{b}R^{c} (XI)

in which
R^{a}, R^{b} and R^{c} are each independently C₁-C₁₈-alkyl, C₂-C₁₈-alkyl which is optionally interrupted by one or more oxygen and/or sulfur atoms and/or one or more substituted or unsubstituted imino groups, C₆-C₁₂-aryl, C₅-C₁₂-cycloalkyl or a five- to six-membered heterocycle having oxygen, nitrogen and/or sulfur atoms, or two of them together form an unsaturated, saturated or aromatic ring optionally interrupted by one or more oxygen and/or sulfur atoms and/or one or more substituted or unsubstituted imino groups, where the radicals mentioned may each be substituted by functional groups, aryl, alkyl, aryloxy, alkyloxy, halogen, heteroatoms and/or heterocycles,
with the proviso that
at least two of the three R^{a}, R^{b} and R^{c} radicals are different and
the R^{a}, R^{b} and R^{c} radicals together have at least 8, preferably at least 10, more preferably at least 12 and most preferably at least 13 carbon atoms.

R^{a}, R^{b} and R^{c} are preferably each independently C₁-C₁₈-alkyl, C₆-C₁₂-aryl or C₅-C₁₂-cycloalkyl, and more preferably C₁-C₁₈-alkyl, where the radicals mentioned may each be substituted by functional groups, aryl, alkyl, aryloxy, alkyloxy, halogen, heteroatoms and/or heterocycles.

Examples of the particular groups have already been listed above.

Preferred definitions of the R^{a}, R^{b} and R^{c} radicals are methyl, ethyl, n-propyl, isopropyl, n-butyl, sec-butyl, tert-butyl, n-pentyl (n-amyl), 2-pentyl (sec-amyl), 3-pentyl, 2,2-dimethylprop-1-yl (neopentyl), n-hexyl, n-heptyl, n-octyl, isooctyl, 2-ethylhexyl, 1,1-dimethylpropyl, 1,1-dimethylbutyl, benzyl, 1-phenylethyl, 2-phenylethyl, α,α-dimethylbenzyl, phenyl, tolyl, xylyl, α-naphthyl, β-naphthyl, cyclopentyl or cyclohexyl.

When two of the R^{a}, R^{b} and R^{c} radicals form a chain, this may, for example, be 1,4-butylene or 1,5-pentylene.

Examples of the tertiary amines of the formula (XI) are diethyl-n-butylamine, diethyl-tert-butylamine, diethyl-n-pentylamine, diethylhexylamine, diethyloctylamine, diethyl-(2-ethylhexyl)amine, di-n-propylbutylamine, di-n-propyl-n-pentylamine, di-n-propylhexylamine, di-n-propyloctylamine, di-n-propyl-(2-ethylhexyl)amine, diisopropylethylamine, diisopropyl-n-propylamine, diisopropylbutylamine, diisopropylpentylamine, diisopropylhexylamine, diisopropyloctylamine, diisopropyl-(2-ethylhexyl)amine, di-n-butylethylamine, di-n-butyl-n-propylamine, di-n-butyl-n-pentylamine, di-n-butylhexylamine, di-n-butyloctylamine, di-n-butyl-(2-ethylhexyl)amine, N-n-butylpyrrolidine, N-sec-butylpyrrolidine, N-tert-butylpyrrolidine, N-n-pentylpyrrolidine, N,N-dimethylcyclohexylamine, N,N-diethylcyclohexylamine, N-di-n-butylcyclohexylamine, N-n-propylpiperidine, N-isopropylpiperidine, N-n-butylpiperidine, N-sec-butylpiperidine, N-tert-butylpiperidine, N-n-pentylpiperidine, N-n-butylmorpholine, N-sec-butylmorpholine, N-tert-butylmorpholine, N-n-pentylmorpholine, N-benzyl-N-ethylaniline, N-benzyl-N-n-propylaniline, N-benzyl-N-isopropylaniline, N-benzyl-N-n-butylaniline, N,N-dimethyl-p-toluidine, N,N-diethyl-p-toluidine, N,N-di-n-butyl-p-toluidine, diethylbenzylamine, di-n-propylbenzylamine, di-n-butylbenzylamine, diethylphenylamine, di-n-propylphenylamine and di-n-butylphenylamine, and also 1,5-diazabicyclo[4.3.0]non-5-ene (DBN).

Preferred tertiary amines (XI) are diisopropylethylamine, diethyl-tert-butylamine, diisopropylbutylamine, di-n-butyl-n-pentylamine, N,N-di-n-butylcyclohexylamine, and tertiary amines of pentyl isomers.

Particularly preferred tertiary amines are di-n-butyl-n-pentylamine and tertiary amines of pentyl isomers.

The melting points of the salts of the particularly preferred auxiliary bases are generally less than 160° C., more preferably less than 100° C. and most preferably less than 80° C.

For example, the hydrochloride of 1-methylimidazole has a melting point of about 75° C., the hydrochloride of 2-ethylpyridine a melting point of about 55° C. The hydrochloride of 1-butylimidazole is liquid even at room temperature.

The free base can be recovered in the manner known to the person skilled in the art from the halide of the auxiliary base which is obtained as the high boiler, and it can be recycled into the process.

This can be done, for example, by releasing the halide of the auxiliary base with a strong base, e.g. NaOH, KOH, Ca(OH)₂, milk of lime, Na₂CO₃, NaHCO₃, K₂CO₃, or KHCO₃, optionally in a solvent, for example water, methanol, ethanol, n- or isopropanol, n-butanol, n-pentanol or butanol or pentanol isomer mixtures, or acetone. The auxiliary base thus released can be removed when it forms a separate phase or, if it is miscible with the salt of the stronger base or the solution of the salt of the stronger base, by distillation out of the mixture. If required, the auxiliary base released can also be removed from the salt of the stronger base or the solution of the salt of the stronger base by extraction with an extractant. Extractants are, for example, solvents, alcohols or amines.

If required, the auxiliary base can be washed with water or aqueous NaCl or Na₂SO₄ solution and then dried, for example by removal of water optionally present with the aid of an azeotropic distillation with benzene, toluene, xylene, butanol or cyclohexane.

If required, the base can be distilled before reuse.

A further means of recycling is to distill the halide of the auxiliary base, in the course of which the salt is cleaved thermally to its starting materials, i.e. the free base and hydrogen halide.

When an inorganic base is used instead of the nonvolatile auxiliary base in the process according to the invention for purifying carboxylic acids comprising halogen compounds, the results obtained, as described above, in the distillation of carboxylic acids in the presence of inorganic bases do not have all the advantages of the process according to the invention. In this procedure, the inorganic bases used are alkali metal and alkaline earth metal hydroxides, for example sodium hydroxide, potassium hydroxide and calcium hydroxide, alkali metal carbonates and alkali metal hydrogencarbonates, and also mixtures of the alkali metal and alkaline earth metal salts mentioned. Preference is given to using aqueous solutions of these inorganic bases, the weight ratio of the inorganic base:water being between 10:90 and 90:10, preferably between 25:75 and 75:25, more preferably between 40:60 and 60:40. Preference is given to alkali metal and alkaline earth metal hydroxides, especially to alkali metal hydroxides, and most preferably to sodium hydroxide and potassium hydroxide.

Useful carboxylic acids comprising halogen compounds include the above-described carboxylic acids contaminated by halogen compounds. The contaminated carboxylic acids may comprise a low boiler, which may include the above-described low boilers in the likewise above-described amounts. In a preferred embodiment of this procedure, a mixture of the carboxylic acid to be purified and of the inorganic base is introduced into a distillation column between the bottom and top thereof, low boilers are drawn off at the top of the distillation column, and pure carboxylic acid is drawn off in gaseous form from the lower region, the bottom or the circulation system of the bottom heater of the distillation column. Useful apparatus for the distillation is the same as described above.

In an embodiment which is simple in terms of apparatus, the mixture of the carboxylic acid to be purified and of the inorganic base is introduced into a distillation column between the top and bottom thereof. The distillation column comprises a column section above the feed, designed as a rectifying section with a plurality of theoretical plates, and a column part below the feed, designed as a stripping section. Low boilers are drawn off at the top of the distillation column, for example as pure low boilers and/or in the form of a low boiler-carboxylic acid azeotrope. The pure carboxylic acid is drawn off in gaseous form from the lower region, the bottom or the circulation system of the bottom heater of the distillation column. The inorganic base and the halide of the base collect in the bottoms.

The mixture of the carboxylic acid to be purified and of the inorganic base can appropriately be converted in a preliminary reactor prior to introduction into the distillation column, in order to complete the reaction between the halogen halide and the base and/or thermal decomposition reactions of organic halogen compounds.

This embodiment is, for example, particularly suitable when the carboxylic acid is acetic acid and the low boiler is heptane (including isomer mixtures).

The process can be executed batchwise or preferably continuously.

The inorganic base binds the halogen halide which is present in the carboxylic acid and/or has been eliminated from halogen compounds by thermal action, forming salts, and in this way lowers the vapor pressure thereof, such that the hydrogen halide is retained in the distillation bottoms and does not pass over into the distillate.

The inorganic base is used in a stoichiometric amount or in a stoichiometric excess (calculated as neutralization equivalents of the base), based on the halogen compounds present in the carboxylic acid to be purified (calculated as halogen atoms). A neutralization equivalent is understood to mean the imaginary fraction of the base that can absorb a proton. The base is generally used in an amount of 1 to 30 equivalents, preferably 1 to 2 equivalents (calculated as neutralization equivalents), based on the halogen compounds present (calculated as halogen atoms). In the case of purification in the presence of inorganic bases, solid deposits which can form in the distillation apparatus can impair disruption-free operation of the distillation apparatus.

The invention is illustrated in detail by the examples which follow. In the examples, all figures in % are understood as % by weight.

### EXAMPLE 1

### Removal of chloride and heptane from acetic acid by means of methylimidazole

### Test 1:

A 250 ml two-neck flask with attached column (15 cm with 8 mm Raschig rings) and water-cooled distillation apparatus was initially charged with 95.00 g of acetic acid (total chlorine 190 ppm; 1.9% heptane), and 5.00 g of methylimidazole (MIA) were added thereto. This heated the flask contents by 6° C. to 31° C. The flask was heated in an oil bath. In the course of distillation under standard pressure, a first fraction (fraction 1, 1.4 g, biphasic) was distilled over at bath temperature (BT) 129-135° C., bottom temperature (ST) up to 119° C. and distillation temperature (DT) up to 80° C. In the course of further heating, the main fraction (fraction 2, monophasic, 69.8 g) distilled over at BT 165° C., ST up to 134° C. and DT up to 95-120° C. In the course of further heating to BT up to 185° C., ST up to 163° C. and DT max. 112° C., a final fraction was obtained (fraction 3, 12.8 g). 15.2 g of bottom residue were obtained.

### Test 2:

Test 1 was repeated, except that 97.50 g of acetic acid were initially charged and 2.50 g of MIA were added. In the course of addition, some fuming and heating by 5° C. to 30° C. were observed. In the course of distillation under standard pressure, a first fraction distilled over at BT 131-142° C., ST up to 118.5° C. and DT up to 81° C. (fraction 1, 3.0 g biphasic). In the course of further heating, the main fraction (fraction 2, monophasic, 83.2 g) distilled over at BT up to 166° C., ST up to 145° C. and DT up to 86-120° C. In the course of further heating to BT up to 185° C., ST up to 167° C. and maximum DT 60° C., a final fraction was obtained (fraction 3, 5.0 g). 9.4 g of bottom residue were obtained.

### Test 3:

Test 1 was repeated, except that 99.00 g of acetic acid were initially charged and 1.00 g of MIA were added. In the course of addition, some fuming and heating by 4° C. to 29° C. were observed. In the course of distillation under standard pressure, a first fraction distilled over at BT 131-146° C., ST up to 117° C. and DT up to 89° C. (fraction 1, 2.9 g biphasic). In the course of further heating, the main fraction (fraction 2, monophasic, 90.2 g) distilled over at BT up to 166° C., ST up to 147° C. and DT up to 116-120° C. In the course of further heating to BT up to 185° C., ST up to 168° C. and maximum DT 86° C., a colorless final fraction was obtained (fraction 3, 2.0 g). 5.7 g of bottom residue were obtained.

The analysis results of tests 1 to 3 are compiled in the following table:

| Sample | Total chlorine | Heptane |
|---|---|---|
| Acetic acid used | 190 ppm | 1.9% |
| MIA used | <0.001% | |
| Test1 Fr 2 | 12 ppm | 0.4% |
| Test2 Fr 2 | 11 ppm | 0.1% |
| Test3 Fr 2 | 10 ppm | 0.1% |

### EXAMPLE 2

### Continuous Removal of Heptane and Chloride from Acetic Acid

An apparatus consisting of two thin-film evaporators with wipers (wiper length 20 cm) was used. A mixing bottle (1000 ml bottle) was initially charged with the acetic acid to be purified by means of a funnel; bottom discharge from the second thin-film evaporator was also recycled via a membrane metering pump. The bottle was shaken by hand in order to mix the contents. Feed from the mixing bottle was fed by means of a membrane metering pump to the first thin-film evaporator which was purged with nitrogen in countercurrent. The distillate was collected below the lateral cooler; the bottoms were passed into a 500 ml two-neck flask which was heated with a thermostat. By means of a membrane metering pump, bottoms discharge from the first thin-film evaporator was passed into the second thin-film evaporator. The distillate was separated by means of a 10 cm column with 8 mm Raschig rings and distilled off by means of a distillation apparatus. The bottoms were passed into a 500 ml two-neck flask which was heated with a thermostat.

### Day 1:

The mixing bottle was initially charged with 976.0 g of acetic acid (total chlorine 0.014%) and 25.4 g of 1-methylimidazole.

Settings of the first thin-film evaporator: BT 118° C., 400 rpm, feed about 0.5 kg/h, nitrogen about 10 l/h.

Settings of the second thin-film evaporator: BT 190° C., 400 rpm, feed about 0.5 kg/h.

In the first thin-film evaporator, a small amount of low boilers distilled off; the biphasic condensate is discarded. After a first runnings period of about 20 min., the bottoms are conveyed directly into the second thin-film evaporator in which acetic acid is obtained as the distillate, which was collected. As soon as an amount of 250-350 ml of bottoms had been attained, the bottoms were conveyed into the mixing bottle (after 1 h 30 min, 2 h 10 min, 2 h 30 min, 2 h 50 min).

Distillate 1 obtained: 456.0 g
Distillate 2 obtained: 437.5 g
Distillate 3 obtained: 454.2 g

### Day 2:

The mixing bottle was initially charged with acetic acid mixed with recycled bottoms from the second thin-film evaporator.

Settings of the first thin-film evaporator: BT 118° C., 400 rpm, feed about 0.5 kg/h, nitrogen about 10 l/h.

Settings of the second thin-film evaporator: BT 195° C., 400 rpm, feed about 0.5 kg/h.

In the first thin-film evaporator, a small amount of low boilers distilled off (31.7 g in total); the biphasic condensate is discarded. After a first runnings time of about 20 min, the bottoms were conveyed directly into the second thin-film evaporator in which acetic acid is obtained as the distillate, which was collected. As soon as an amount of 250-350 ml of bottoms had been attained, the bottoms were conveyed into the mixing bottle (after 1 h 25 min, then ever more rapidly at 30-15 minute intervals).

Distillate 4 obtained: 409.5 g
Distillate 5 obtained: 457.3 g
Distillate 6 obtained: 400.4 g
Distillate 7 obtained: 283.0 g

After the withdrawal of distillate had ended, 353.2 g of bottoms remained.

The analysis results of distillates 1 to 7 are compiled in the following table:

| Sample | Total chlorine |
|---|---|
| Acetic acid used | 0.014% |
| Distillate 1 | 18 ppm |
| Distillate 2 | 25 ppm |
| Distillate 3 | 19 ppm |
| Distillate 4 | 22 ppm |
| Distillate 5 | 15 ppm |
| Distillate 6 | 18 ppm |
| Distillate 7 | 18 ppm |

### EXAMPLE 3

### Continuous Removal of Heptane and Chloride from Acetic Acid

The apparatus used consisted of a preliminary reactor and a bubble-cap tray column with 8 trays. The preliminary reactor used was a 500 ml standard reactor with overflow and glass stirrer stirring at 50 rpm. The acetic acid was fed in by means of a membrane metering pump with Teflon internals; the base was fed in by means of a piston metering pump with Teflon internals.

The standard bubble-cap tray column used had a total of 8 bubble-cap trays and was heated with electrical heating jackets. The bottom used was a 2.5 l standard reactor with two standard ground-glass joints, a glass stirrer and fill level monitoring, which was heated with a thermostat. Vacuum was generated by a regulated oil pump.

The feed from the preliminary reactor was conducted to tray 4 of the bubble-cap tray column. Vapor was drawn off through the second standard ground-glass joint of the bottom reactor lid. The bottoms were condensed in a jacket coil condenser. The condensate was conveyed out with a membrane metering pump. In order to eliminate partial pressure differences, the top of the condenser was connected directly to tray 6 (counted from the bottom) via a PVC hose and an installed needle valve. In order to keep the condensate fill level constant, excess distillate was conveyed directly into the bottom via a bypass incorporating a membrane metering pump.

Via a heated bridge, the low boiler mixture was drawn off at the top of the column, condensed in a condenser and introduced into a magnetic liquid distributor which was set at a ratio of 99:1. The larger stream was conveyed back to the uppermost tray of the column by means of a level-regulated membrane metering pump; the smaller stream was withdrawn by means of a level-controlled membrane metering pump.

At the start of the experiment, the bottom vessel was initially charged with 1010 g of acetic acid and 2.1 g of 1-methylimidazole (MIA). 520 g/h of acetic acid and 1.05 g/h of MIA were metered into the preliminary reactor.

A vacuum of 515 mbar was established. The bottom heater was set to 139° C. and was increased stepwise to 144° C.; the heating bands were set to 100° C./90° C./60° C./40° C. The temperature of the preliminary reactor was 78° C., the temperature of the condenser 94° C. 480 g/h of pure acetic acid were withdrawn.

The total chlorine content of the acetic acid used was 0.07%. In the resulting pure acetic acid, the total chlorine content was less than 10 ppm. The chloride ion content in the pure acetic acid was determined to be 4 ppm. The heptane content was reduced from 1% to 0.1%.

### EXAMPLE 4

### Continuous Removal of Heptane and Chloride from Acetic Acid

As in example 3, the apparatus used consisted of a preliminary reactor and a bubble-cap tray column with 8 trays. The preliminary reactor is identical to example 3. The acetic acid was blended with the base beforehand in a bottle. The acetic acid-base mixture was fed in by means of a membrane metering pump with Teflon internals. The standard bubble-cap tray column used is identical to example 3; the bottom vessel and the vacuum also correspond to example 3.

The feed from the preliminary reactor was conducted to tray 4 of the bubble-cap tray column. Vapor was drawn off through the second standard ground-glass joint of the bottom reactor lid. A single bubble-cap tray was connected upstream of the jacketed coil condenser. The vapor was condensed in the jacketed coil condenser. The condensate was conveyed out with a membrane metering pump. In order to eliminate partial pressure differences, the top of the condenser was connected directly to tray 3 (counted from the bottom) by means of a Teflon hose and an installed needle valve. The low boiler removal is as described in example 3.

At the start of the experiment, the bottom vessel was initially charged with 999.1 g of acetic acid and 0.45 g of 1-methylimidazole. 520 g/h of acetic acid/methylimidazole mixture (with a methylimidazole to acetic acid ratio of 0.45 g to 1 kg) were metered into the preliminary reactor.

A vacuum of 515 mbar was established. The bottom heater was set to 145° C.; the heating bands were set to 100° C./90° C./60° C./40° C. The temperature of the preliminary reactor was 78° C., the temperature of the condenser 94° C. 480 g/h of pure acetic acid were withdrawn.

The total chlorine content of the acetic acid used was 0.013%, 50 ppm of which was in the form of chloride. In the pure acetic acid obtained, the total chlorine content was less than 3 ppm. The heptane content was reduced from 1% to 0.1%.

### COMPARATIVE EXAMPLE 1

### Continuous Removal of Heptane and Chloride from Acetic Acid with the Aid of Sodium Hydroxide

The apparatus used consisted of a preliminary reactor and a bubble-cap tray column with 8 trays. The preliminary reactor used was a 500 ml standard reactor with overflow and glass stirrer stirring at 50 rpm. The acetic acid was fed in by means of a membrane metering pump with Teflon internals; the base was fed in by means of a piston metering pump with Teflon internals.

The standard bubble-cap tray column used had a total of 8 bubble-cap trays and was heated with electrical heating jackets. The bottom used was a 2.5 l standard reactor with two standard ground-glass joints, a glass stirrer and fill level monitoring, which was heated with a thermostat. Vacuum was generated by a regulated oil pump. The feed from the preliminary reactor was conducted to tray 4 of the bubble-cap tray column. Vapor was drawn off through the second standard ground-glass joint of the bottom reactor lid. The vapor was condensed in a jacketed coil condenser. The condensate was conveyed out with a membrane metering pump. In order to eliminate partial pressure differences, the top of the condenser was connected directly to tray 3 (counted from the bottom) via a PVC hose and an installed needle valve. In order to keep the condensate fill level constant, excess distillate was conveyed directly into the bottom vessel via a bypass incorporating a membrane metering pump.

Via a heated bridge, the low boiler mixture was drawn off at the top of the column, condensed in a condenser and introduced into a magnetic liquid distributor which was set at a ratio of 99:1. The larger stream was conveyed back to the uppermost tray of the column by means of a level-regulated membrane metering pump; the smaller stream was withdrawn by means of a level-regulated membrane metering pump.

At the start of the experiment, the bottom vessel was initially charged with 999.6 g of acetic acid and 0.4 g of 50% sodium hydroxide solution. 520 g/h of acetic acid-NaOH mixture were metered into the preliminary reactor in the same ratio.

A vacuum of 505 mbar was established. The bottom heater was set to 145° C.; the heating bands were set to 100° C./90° C./60° C./40° C. The temperature of the preliminary reactor was 78° C., the temperature of the condenser 94° C. 480 g/h of pure acetic acid were withdrawn.

The total chlorine content of the acetic acid used was 0.015%. In the resulting pure acetic acid, the total chlorine content was 5 ppm in the first five liters, then not more than 3 ppm. The heptane content was reduced from 0.9% to from 0.11 to 0.05%.

### COMPARATIVE EXAMPLE 2

### Continuous Removal of Heptane and Chloride from Acetic Acid with the Aid of Potassium Hydroxide

As in example 1, the apparatus used consisted of a preliminary reactor and a bubble-cap tray column with 8 trays. The preliminary reactor is identical to comparative example 1. The acetic acid was blended with the base beforehand. The acetic acid was fed in by means of a membrane metering pump with Teflon internals; the base was fed in by means of a piston metering pump with Teflon internals.

The standard bubble-cap tray column used is identical to comparative example 1; the bottom vessel and the vacuum also correspond to comparative example 1.

The feed from the preliminary reactor was conducted to tray 4 of the bubble-cap tray column. Vapor was drawn off through the second standard ground-glass joint of the bottom reactor lid. The vapor was condensed in a jacketed coil condenser. The condensate was conveyed out with a membrane metering pump. In order to eliminate partial pressure differences, the top of the condenser was connected directly to tray 2 (counted from the bottom) via a PVC hose and an installed needle valve. In order to keep the condensate fill level constant, excess distillate was conveyed directly into the bottom vessel via a bypass incorporating a membrane metering pump.

Via a heated bridge, the low boiler mixture was drawn off at the top of the column, condensed in a condenser and introduced into a magnetic liquid distributor which was set at a ratio of 99:1. The larger stream was conveyed back to the uppermost tray of the column by means of a level-regulated membrane metering pump; the smaller stream was withdrawn by means of a level-regulated membrane metering pump.

At the start of the experiment, the bottom vessel was initially charged with 990.5 g of acetic acid and 9.5 g of 50% potassium hydroxide solution. 520 g/h of acetic acid-KOH mixture were metered into the preliminary reactor in the same ratio.

A vacuum of 505 mbar was established. The bottom heater was set to 145° C.; the heating bands were set to 100° C./90° C./60° C./40° C. The temperature of the preliminary reactor was 78° C., the temperature of the condenser 94° C. 480 g/h of pure acetic acid were withdrawn.

The total chlorine content of the acetic acid used was 0.01%. In the resulting pure acetic acid, the total chlorine content was less than 3 ppm. The heptane content was reduced from 0.9% to 0.05%.

1.-11. (canceled)

12. A process for purifying carboxylic acids comprising halogen compounds, comprising distilling the carboxylic acid in the presence of a nonvolatile auxiliary base, the halide of which is liquid at the boiling temperature of the carboxylic acid.

13. The process according to claim 12, wherein the halogen compounds are selected from chlorine compounds and bromine compounds.

14. The process according to claim 13, wherein the halogen compounds are chlorine compounds.

15. The process according to claim 12, wherein the halogen compounds comprise hydrogen halide.

16. The process according to claim 12, wherein the auxiliary base is used in an amount of 1 to 30 equivalents, calculated as neutralization equivalents, based on the halogen compounds present in the carboxylic acid to be purified, calculated as halogen atoms.

17. The process according to claim 12, wherein the carboxylic acid is selected from formic acid and acetic acid.

18. The process according to claim 12, wherein the carboxylic acid further comprises at least one low boiler.

19. The process according to claim 18, wherein a mixture of the carboxylic acid to be purified and of the auxiliary base is introduced into a distillation column between the top and bottom thereof, low boilers are drawn off at the top of the distillation column and pure carboxylic acid is drawn off in gaseous form from the lower region, the bottom or the circulation of the bottom heater of the distillation column.

20. The process according to claim 18, wherein the carboxylic acid is acetic acid and the low boiler is heptane.

21. The process according to claim 12, wherein the auxiliary base is selected from the formulae (Ia) to (Ir) 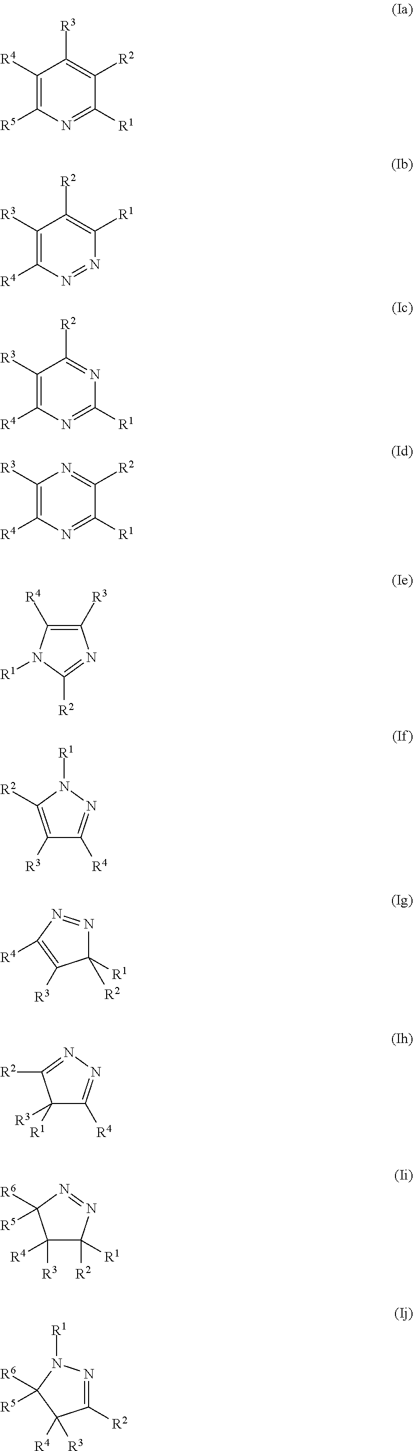 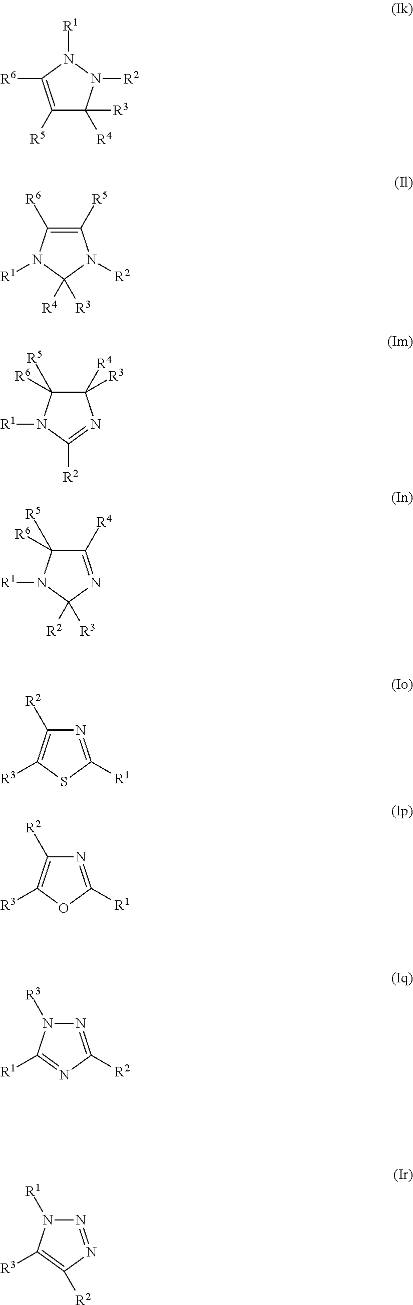
wherein
R¹, R², R³, R⁴, R⁵ and R⁶ are each independently hydrogen, C₁-C₁₈-alkyl, C₂-C₁₈-alkyl which is optionally interrupted by one or more oxygen and/or sulfur atoms and/or one or more substituted or unsubstituted imino groups, C₆-C₁₂-aryl, C₅-C₁₂-cycloalkyl or a five- to six-membered heterocycle having oxygen, nitrogen and/or sulfur atoms, where the radicals mentioned may each be substituted by functional groups, aryl, alkyl, aryloxy, alkyloxy, halogen, heteroatoms and/or heterocycles.

22. The process according to claim 12, wherein the auxiliary base is 1-n-butylimidazole, 1-methylimidazole, 2-methylpyridine or 2-ethylpyridine.
